(19) United States
(12) Patent Application Publication
(10) Pub. No.: US 2023/0280243 A1
(43) Pub. Date: Sep. 7, 2023

(54) OPTICAL BACKGROUND SUPPRESSION IN BINDING ASSAYS THAT EMPLOY POLYMERIC MICROSPHERES

(71) Applicant: PNP Research LLC, Drury, MA (US)

(72) Inventors: Yolanda Giménez Molina, Monóvar (ES); Germán González Serrano, Sant Joan D´Alacant (ES); William Peter Hansen, Drury, MA (US)

(21) Appl. No.: 17/957,192

(22) Filed: Sep. 30, 2022

Related U.S. Application Data
(60) Provisional application No. 63/251,225, filed on Oct. 1, 2021.

(51) Int. Cl. G01N 1/40; G01N 33/543

(52) U.S. Cl. CPC G01N 1/4077; G01N 33/54346

(57) ABSTRACT

Methods are herein described which reduce the non-specific light scattering background from polymeric microspheres in aqueous suspension to a level where individual metal or metal-like plasmonic nanoparticles that have been chemically bound to the surface of individual polymeric microspheres can be imaged by high resolution optics and enumerated by image analysis software. The non-specific light scattering background is reduced by employing aqueous-miscible solvents to remove residual water at the microsphere surfaces and resuspending the microspheres in a fluid having a refractive index substantially similar to the refractive index of polymeric microspheres. Embodiments of the method disclosed herein enable sensitive quantitative assays for ligand receptor binding, antigen antibody binding and nucleic acid hybridization binding. The embodiments disclosed herein further may employ polystyrene microspheres and gold plasmonic nanoparticles.

### CROSS-REFERENCE TO RELATED APPLICATIONS

This application claims priority under 35 U.S.C. § 119(e) to U.S. Provisional Application Ser. No. 63/251,225, titled “OPTICAL BACKGROUND SUPPRESSION IN BINDING ASSAYS THAT EMPLOY POLYMERIC MICROSPHERES,” filed on Oct. 1, 2021, which is hereby incorporated by reference in its entirety.

### FIELD

This disclosure concerns the sensitive optical imaging and quantitative enumeration of plasmonic nanoparticles that bind to the surface of polymeric microspheres in biochemical reactions. The embodiments of this disclosure enable sensitive quantitative assays for ligand receptor binding, antigen antibody binding and nucleic acid hybridization binding. Specifically, this disclosure provides for a reduction in the non-specific light scattering background from polymeric microspheres to a level where individual nanoparticles that are bound to the surface of individual polymeric microspheres can be imaged by high resolution optics and enumerated by image analysis software.

### SUMMARY OF THE INVENTION

In accordance with an aspect, there is provided a method of preparing microspheres. The method may include providing an aqueous suspension of the microspheres. The method may include concentrating the microspheres to form a first portion of concentrated microspheres and an aqueous supernatant. The method further may include removing the aqueous supernatant from the first portion of the concentrated microspheres. The method further may include suspending the first portion of the concentrated microspheres in a solvent miscible with the aqueous supernatant to form solvent-suspended microspheres. The method additionally may include concentrating the solvent-suspended microspheres to form a second portion of concentrated microspheres and a solvent supernatant. The method further may include removing the solvent supernatant from the second portion of concentrated microspheres, thereby preparing the microspheres.

In further embodiments, the method may include suspending the second portion of concentrated microspheres in a fluid having a refractive index substantially similar to that of a material used to manufacture the microspheres. This fluid may include, but is not limited to, an oil, an ester, an aromatic liquid, a non-polar solvent, or a polyepoxide. In specific embodiments, the suspending fluid for the second portion of concentrated microspheres is cinnamon bark oil.

In some embodiments, concentrating the microspheres or the solvent-suspended microspheres occurs under centrifugation conditions. The centrifugation occurs for about 1 second to about 20 minutes, e.g., about 1 second to about 20 seconds, about 5 seconds to about 30 seconds, about 10 seconds to about 40 seconds, about 30 seconds to about 1 minute, about 1 minute to about 20 minutes, about 2 minutes to about 18 minutes, about 3 minutes to about 17 minutes, about 4 minutes to about 16 minutes, about 5 minutes to about 15 minutes, e.g., about 6 minutes to about 14 minutes, about 7 minutes to about 13 minutes, about 8 minutes to about 12 minutes, about 9 minutes to about 11 minutes, or about 10 minutes, e.g., about 1 second, about 2 seconds, about 3 seconds, about 4 seconds, about 5 seconds, about 6 seconds, about 7 seconds, about 8 seconds, about 9 seconds, about 10 seconds, about 11 seconds, about 12 seconds, about 13 seconds, about 14 seconds, about 15 seconds, about 16 seconds, about 17 seconds, about 18 seconds, about 19 seconds, about 20 seconds, about 21 seconds, about 22 seconds, about 23 seconds, about 24 seconds, about 25 seconds, about 26 seconds, about 27 seconds, about 28 seconds, about 29 seconds, about 30 seconds, about 31 seconds, about 32 seconds, about 33 seconds, about 34 seconds, about 35 seconds, about 36 seconds, about 37 seconds, about 38 seconds, about 39 seconds, about 40 seconds, about 41 seconds, about 42 seconds, about 43 seconds, about 44 seconds, about 45 seconds, about 46 seconds, about 47 seconds, about 48 seconds, about 49 seconds, about 50 seconds, about 51 seconds, about 52 seconds, about 53 seconds, about 54 seconds, about 55 seconds, about 56 seconds, about 57 seconds, about 58 seconds, about 59 seconds, about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, or about 20 minutes.

In some embodiments, the provided microspheres may have a diameter from about 1 μm to about 100 μm, e.g., about 1 μm to about 100 μm, about 2 μm to about 90 μm, about 3 μm to about 80 μm, about 4 μm to about 70 μm, about 5 μm to about 60 μm, about 6 μm to about 50 μm, about 7 μm to about 40 μm, about 8 μm to about 30 μm, about 9 μm to about 20 μm, or about 10 μm, e.g., about 1 μm, about 2 μm, about 3 μm, about 4 μm, about 5 μm, about 6 μm, about 7 μm, about 8 μm, about 9 μm, about 10 μm, about 11 μm, about 12 μm, about 13 μm, about 14 μm, about 15 μm, about 16 μm, about 17 μm, about 18 μm, about 19 μm, about 20 μm, about 21 μm, about 22 μm, about 23 μm, about 24 μm, about 25 μm, about 26 μm, about 27 μm, about 28 μm, about 29 μm, about 30 μm, about 31 μm, about 32 μm, about 33 μm, about 34 μm, about 35 μm, about 36 μm, about 37 μm, about 38 μm, about 39 μm, about 40 μm, about 41 μm, about 42 μm, about 43 μm, about 44 μm, about 45 μm, about 46 μm, about 47 μm, about 48 μm, about 49 μm, about 50 μm, about 51 μm, about 52 μm, about 53 μm, about 54 μm, about 55 μm, about 56 μm, about 57 μm, about 58 μm, about 59 μm, about 60 μm, about 61 μm, about 62 μm, about 63 μm, about 64 μm, about 65 μm, about 66 μm, about 67 μm, about 68 μm, about 69 μm, about 70 μm, about 71 μm, about 72 μm, about 73 μm, about 74 μm, about 75 μm, about 76 μm, about 77 μm, about 78 μm, about 79 μm, about 80 μm, about 81 μm, about 82 μm, about 83 μm, about 84 μm, about 85 μm, about 86 μm, about 87 μm, about 88 μm, about 89 μm, about 90 μm, about 91 μm, about 92 μm, about 93 μm, about 94 μm, about 95 μm, about 96 μm, about 97 μm, about 98 μm, about 99 μm, or 100 μm in diameter.

In some embodiments, the provided microspheres include surface-bound nanoparticles. In some embodiments, the provided microspheres include surface-bound fluorochromes. In some embodiments, the provided microspheres include embedded fluorochromes.

In some embodiments, the surface-bound nanoparticles of the provided microspheres have a diameter from about 40 nm to about 150 nm, e.g., about 40 nm to about 60 nm, about 45 nm to about 55 nm, or about 50 nm, about 50 nm to about 100 nm, about 60 nm to about 90 nm, about 70 nm to about 80 nm, about 100 nm to about 130 nm, or about 120 nm to about 150 nm, e.g., about 40 nm, about 41 nm, about 42 nm, about 43 nm, about 44 nm, about 45 nm, about 46 nm, about 47 nm, about 48 nm, about 49 nm, about 50 nm, about 51 nm, about 52 nm, about 53 nm, about 54 nm, about 55 nm, about 56 nm, about 57 nm, about 58 nm, about 59 nm, about 60 nm, about 61 nm, about 62 nm, about 63 nm, about 64 nm, about 65 nm, about 66 nm, about 67 nm, about 68 nm, about 69 nm, about 70 nm, about 71 nm, about 72 nm, about 73 nm, about 74 nm, about 75 nm, about 76 nm, about 77 nm, about 78 nm, about 79 nm, about 80 nm, about 81 nm, about 82 nm, about 83 nm, about 84 nm, about 85 nm, about 86 nm, about 87 nm, about 88 nm, about 89 nm, about 90 nm, about 91 nm, about 92 nm, about 93 nm, about 94 nm, about 95 nm, about 96 nm, about 97 nm, about 98 nm, about 99 nm, about 100 nm in diameter. In some embodiments, the nanoparticles can have a diameter of between 100 nm and 150 nm, e.g., about 101 nm, about 102 nm, about 103 nm, about 104 nm, about 105 nm, about 106 nm, about 107 nm, about 108 nm, about 109 nm, about 110 nm, about 111 nm, about 112 nm, about 113 nm, about 114 nm, about 115 nm, about 116 nm, about 117 nm, about 118 nm, about 119 nm, about 120 nm, about 121 nm, about 122 nm, about 123 nm, about 124 nm, about 125 nm, about 126 nm, about 127 nm, about 128 nm, about 129 nm, about 130 nm, about 131 nm, about 132 nm, about 133 nm, about 134 nm, about 135 nm, about 136 nm, about 137 nm, about 138 nm, about 139 nm, about 140 nm, about 141 nm, about 142 nm, about 143 nm, about 144 nm, about 145 nm, about 146 nm, about 147 nm, about 148 nm, about 149 nm, or about 150 nm.

In some embodiments, providing the aqueous suspension of the microspheres may include providing an aqueous suspension of polystyrene microspheres.

In some embodiments, suspending the first portion of the concentrated microspheres comprises suspending the first portion of the concentrated microspheres in a solvent selected from alcohols, ketones, glycol ethers, alkoxy-substituted hydrocarbons, and esters. For example, suitable solvents for suspending the first portion of the concentrated microspheres include alcohols, specifically ethanol.

In accordance with an aspect, there is provided a method of preparing microspheres. The method may include providing an aqueous suspension of the microspheres. The method may include concentrating the microspheres to form a first portion of concentrated microspheres and an aqueous supernatant. The method further may include removing the aqueous supernatant from the first portion of the concentrated microspheres. The method further may include suspending the first portion of the concentrated microspheres in a solvent miscible with the aqueous supernatant to form solvent-suspended microspheres. The method additionally may include concentrating the solvent-suspended microspheres to form a second portion of concentrated microspheres and a solvent supernatant. The method further may include removing the solvent supernatant from the second portion of concentrated microspheres, thereby preparing the microspheres. The method additionally may include suspending the second portion of concentrated microspheres in a fluid having a refractive index substantially similar to that of a material used to manufacture the microspheres.

For example, the fluid used to suspend the second portion of concentrated microspheres may include, but is not limited to, an oil, an ester, an aromatic liquid, a non-polar solvent, or a polyepoxide. In specific embodiments, the suspending fluid for the second portion of concentrated microspheres is cinnamon bark oil.

In some embodiments, concentrating the microspheres or the solvent-suspended microspheres occurs under centrifugation conditions. The centrifugation occurs for about 1 second to about 20 minutes, e.g., about 1 second to about 20 seconds, about 5 seconds to about 30 seconds, about 10 seconds to about 40 seconds, about 30 seconds to about 1 minute, about 1 minute to about 20 minutes, about 2 minutes to about 18 minutes, about 3 minutes to about 17 minutes, about 4 minutes to about 16 minutes, about 5 minutes to about 15 minutes, e.g., about 6 minutes to about 14 minutes, about 7 minutes to about 13 minutes, about 8 minutes to about 12 minutes, about 9 minutes to about 11 minutes, or about 10 minutes, e.g., about 1 second, about 2 seconds, about 3 seconds, about 4 seconds, about 5 seconds, about 6 seconds, about 7 seconds, about 8 seconds, about 9 seconds, about 10 seconds, about 11 seconds, about 12 seconds, about 13 seconds, about 14 seconds, about 15 seconds, about 16 seconds, about 17 seconds, about 18 seconds, about 19 seconds, about 20 seconds, about 21 seconds, about 22 seconds, about 23 seconds, about 24 seconds, about 25 seconds, about 26 seconds, about 27 seconds, about 28 seconds, about 29 seconds, about 30 seconds, about 31 seconds, about 32 seconds, about 33 seconds, about 34 seconds, about 35 seconds, about 36 seconds, about 37 seconds, about 38 seconds, about 39 seconds, about 40 seconds, about 41 seconds, about 42 seconds, about 43 seconds, about 44 seconds, about 45 seconds, about 46 seconds, about 47 seconds, about 48 seconds, about 49 seconds, about 50 seconds, about 51 seconds, about 52 seconds, about 53 seconds, about 54 seconds, about 55 seconds, about 56 seconds, about 57 seconds, about 58 seconds, about 59 seconds, about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, or about 20 minutes.

In some embodiments, the provided microspheres may have a diameter from about 1 μm to about 100 μm, e.g., about 1 μm to about 100 μm, about 2 μm to about 90 μm, about 3 μm to about 80 μm, about 4 μm to about 70 μm, about 5 μm to about 60 μm, about 6 μm to about 50 μm, about 7 μm to about 40 μm, about 8 μm to about 30 μm, about 9 μm to about 20 μm, or about 10 μm, e.g., about 1 μm, about 2 μm, about 3 μm, about 4 μm, about 5 μm, about 6 μm, about 7 μm, about 8 μm, about 9 μm, about 10 μm, about 11 μm, about 12 μm, about 13 μm, about 14 μm, about 15 μm, about 16 μm, about 17 μm, about 18 μm, about 19 μm, about 20 μm, about 21 μm, about 22 μm, about 23 μm, about 24 μm, about 25 μm, about 26 μm, about 27 μm, about 28 μm, about 29 μm, about 30 μm, about 31 μm, about 32 μm, about 33 μm, about 34 μm, about 35 μm, about 36 μm, about 37 μm, about 38 μm, about 39 μm, about 40 μm, about 41 μm, about 42 μm, about 43 μm, about 44 μm, about 45 μm, about 46 μm, about 47 μm, about 48 μm, about 49 μm, about 50 μm, about 51 μm, about 52 μm, about 53 μm, about 54 μm, about 55 μm, about 56 μm, about 57 μm, about 58 μm, about 59 μm, about 60 μm, about 61 μm, about 62 μm, about 63 μm, about 64 μm, about 65 μm, about 66 μm, about 67 μm, about 68 μm, about 69 μm, about 70 μm, about 71 μm, about 72 μm, about 73 μm, about 74 μm, about 75 μm, about 76 μm, about 77 μm, about 78 μm, about 79 μm, about 80 μm, about 81 μm, about 82 μm, about 83 μm, about 84 μm, about 85 μm, about 86 μm, about 87 μm, about 88 μm, about 89 μm, about 90 μm, about 91 μm, about 92 μm, about 93 μm, about 94 μm, about 95 μm, about 96 μm, about 97 μm, about 98 μm, about 99 μm, or 100 μm in diameter.

In some embodiments, the provided microspheres include surface-bound nanoparticles. In some embodiments, the provided microspheres include surface-bound fluorochromes. In some embodiments, the provided microspheres include embedded fluorochromes.

In some embodiments, the surface-bound nanoparticles of the provided microspheres have a diameter from about 40 nm to about 150 nm, e.g., about 40 nm to about 60 nm, about 45 nm to about 55 nm, or about 50 nm, about 50 nm to about 100 nm, about 60 nm to about 90 nm, about 70 nm to about 80 nm, about 100 nm to about 130 nm, or about 120 nm to about 150 nm, e.g., about 40 nm, about 41 nm, about 42 nm, about 43 nm, about 44 nm, about 45 nm, about 46 nm, about 47 nm, about 48 nm, about 49 nm, about 50 nm, about 51 nm, about 52 nm, about 53 nm, about 54 nm, about 55 nm, about 56 nm, about 57 nm, about 58 nm, about 59 nm, about 60 nm, about 61 nm, about 62 nm, about 63 nm, about 64 nm, about 65 nm, about 66 nm, about 67 nm, about 68 nm, about 69 nm, about 70 nm, about 71 nm, about 72 nm, about 73 nm, about 74 nm, about 75 nm, about 76 nm, about 77 nm, about 78 nm, about 79 nm, about 80 nm, about 81 nm, about 82 nm, about 83 nm, about 84 nm, about 85 nm, about 86 nm, about 87 nm, about 88 nm, about 89 nm, about 90 nm, about 91 nm, about 92 nm, about 93 nm, about 94 nm, about 95 nm, about 96 nm, about 97 nm, about 98 nm, about 99 nm, about 100 nm in diameter. In some embodiments, the nanoparticles can have a diameter of between 100 nm and 150 nm, e.g., about 101 nm, about 102 nm, about 103 nm, about 104 nm, about 105 nm, about 106 nm, about 107 nm, about 108 nm, about 109 nm, about 110 nm, about 111 nm, about 112 nm, about 113 nm, about 114 nm, about 115 nm, about 116 nm, about 117 nm, about 118 nm, about 119 nm, about 120 nm, about 121 nm, about 122 nm, about 123 nm, about 124 nm, about 125 nm, about 126 nm, about 127 nm, about 128 nm, about 129 nm, about 130 nm, about 131 nm, about 132 nm, about 133 nm, about 134 nm, about 135 nm, about 136 nm, about 137 nm, about 138 nm, about 139 nm, about 140 nm, about 141 nm, about 142 nm, about 143 nm, about 144 nm, about 145 nm, about 146 nm, about 147 nm, about 148 nm, about 149 nm, or about 150 nm.

In some embodiments, providing the aqueous suspension of the microspheres may include providing an aqueous suspension of polystyrene microspheres.

In some embodiments, suspending the first portion of the concentrated microspheres comprises suspending the first portion of the concentrated microspheres in a solvent selected from alcohols, ketones, glycol ethers, alkoxy-substituted hydrocarbons, and esters. For example, suitable solvents for suspending the first portion of the concentrated microspheres include alcohols, specifically ethanol.

### BRIEF DESCRIPTION OF THE DRAWINGS

Various aspects of one or more embodiments are discussed below with reference to the accompanying figures, which are not intended to be drawn to scale. The figures are included to provide an illustration and a further understanding of the various aspects and embodiments. The figures are incorporated in and constitute a part of this specification. But the figures are not intended as a definition of the limits of any particular embodiment. The drawings, together with the remainder of the specification, serve to explain principles and operations of the described and claimed aspects and embodiments. For purposes of clarity, not every component may be labelled in every figure. In the figures:

FIG. 1 illustrates a flowchart of a method of preparing microspheres, according to one embodiment;

FIG. 2 illustrates microscopy images of polystyrene microspheres having a film of liquid water on their outer surfaces and the resulting light scattering pattern;

FIG. 3 illustrates microscopy images of the polystyrene microspheres with the film of liquid water removed by the process of FIG. 1;

FIG. 4 illustrates a polystyrene bead with surface bound plasmonic gold nanoparticles treated by the method of FIG. 1 and imaged with darkfield microscopy at different Z-planes;

FIG. 5 illustrates a time-series analysis of a polystyrene bead with surface bound plasmonic gold nanoparticles imaged at different times after fabrication;

FIGS. 6A-6B illustrates polystyrene beads with surface bound plasmonic gold nanoparticles bound at different surface densities. FIG. 6A illustrates a polystyrene bead with surface bound plasmonic gold nanoparticles prepared from a binding reaction having a nanoparticle to bead ratio of 1000:1. FIG. 6B illustrates a polystyrene bead with surface bound plasmonic gold nanoparticles prepared from a binding reaction having a nanoparticle to bead ratio of 2000:1; and

FIGS. 7A-7B illustrate a polystyrene bead with surface bound plasmonic gold nanoparticles that is also labeled with a fluorescent color and imaged using darkfield and fluorescence microscopy. FIG. 7A illustrates the darkfield microscope image. FIG. 7B illustrates the fluorescence image.

### DETAILED DESCRIPTION

Monoclonal antibodies can be created that bind to specific antigenic structures located on the surface of biological cells. Fluorescent molecules are commonly attached to such antibodies and used as optical markers denoting the presence of specific antigens on the cell surface. This procedure, which is called immunophenotyping, is used in research and hospital flow cytometry or optical microscopy laboratories to identify cell types and function. The detection sensitivity of this method is oftentimes limited by the background fluorescence of the cell itself. Overcoming background fluorescence and enabling reliable cell surface antigen quantification can require on the order of a thousand bound fluorescent markers per cell or more.

It has been demonstrated that plasmonic nanoparticles, such as those made from gold or silver with diameters of approximately 50-100 nanometers (nm), can be employed to replace said fluorescent molecules and create a light scatter based optical signal from individual plasmonic nanoparticles that is orders of magnitude brighter than fluorescence. With most cells, the background light scatter from the cell itself is low compared to the local light scatter signal from a bound plasmonic nanoparticle, and it is possible to reliably locate as few as one bound plasmonic nanoparticle per cell. This brighter signal enables high resolution, three-dimensional, dark field optical imaging of the location of individual plasmonic nanoparticles on the cell surface Image analysis algorithms can be used to enumerate these locations and provide a biologically useful, quantitative measure of the number of antigens per cell. This level of sensitivity enables near single binding site detection on the surface of circulating immune cells. There is a need to achieve this level of sensitivity in other binding assays. These assays include, but are not limited to, ligand receptor binding, antigen antibody binding, nucleic acid hybridization binding, and quality control assays for plasmonic nanoparticle detection of cell surface antigens. One approach to achieving these assays concerns the use of polymeric microspheres in conjunction with plasmonic nanoparticles. In this approach, plasmonic nanoparticles and polymeric microspheres are separately coated with chemical agents that induce a desired binding reaction. Polymeric microspheres can be surface modified and coated with binding agents such as biotin or streptavidin and, alternatively, other passively or covalently immobilized binding agents. However, unlike immune cells, polymeric, e.g., polystyrene, microspheres exhibit a high level of background light scatter which, when imaged under the microscope, can obscure the light scatter from plasmonic nanoparticles bound to its surface and prevent sensitive enumeration of low levels of plasmonic nanoparticle binding.

Under ambient conditions and light agitation, the binding reaction between the binding agents on the surface of the microspheres and the targets to be bound by the binding agents generally occurs over long timescale, e.g., at least 60 minutes, e.g., between 60 to 120 minutes. The long timescale of typical binding reactions has limited the utility of the binding reaction as an assay, e.g., a rapid assay. Increasing the collision frequency of the binding agents on the surface of the microspheres and the targets to be bound by the binding agents mechanically, e.g., using centrifugation, can reduce the binding reaction timescale to about 1 second to about 20 minutes, e.g., about 1 second to about 20 seconds, about 5 seconds to about 30 seconds, about 10 seconds to about 40 seconds, about 30 seconds to about 1 minute, about 1 minute to about 20 minutes, about 2 minutes to about 18 minutes, about 3 minutes to about 17 minutes, about 4 minutes to about 16 minutes, about 5 minutes to about 15 minutes, e.g., about 6 minutes to about 14 minutes, about 7 minutes to about 13 minutes, about 8 minutes to about 12 minutes, about 9 minutes to about 11 minutes, or about 10 minutes, e.g., about 1 second, about 2 seconds, about 3 seconds, about 4 seconds, about 5 seconds, about 6 seconds, about 7 seconds, about 8 seconds, about 9 seconds, about 10 seconds, about 11 seconds, about 12 seconds, about 13 seconds, about 14 seconds, about 15 seconds, about 16 seconds, about 17 seconds, about 18 seconds, about 19 seconds, about 20 seconds, about 21 seconds, about 22 seconds, about 23 seconds, about 24 seconds, about 25 seconds, about 26 seconds, about 27 seconds, about 28 seconds, about 29 seconds, about 30 seconds, about 31 seconds, about 32 seconds, about 33 seconds, about 34 seconds, about 35 seconds, about 36 seconds, about 37 seconds, about 38 seconds, about 39 seconds, about 40 seconds, about 41 seconds, about 42 seconds, about 43 seconds, about 44 seconds, about 45 seconds, about 46 seconds, about 47 seconds, about 48 seconds, about 49 seconds, about 50 seconds, about 51 seconds, about 52 seconds, about 53 seconds, about 54 seconds, about 55 seconds, about 56 seconds, about 57 seconds, about 58 seconds, about 59 seconds, about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, or about 20 minutes.

Transparent microspheres can act as spherical lenses, scattering incident visible light approximately following the laws of ray optics. Darkfield microscopy is a microscopy technique that measures the light scattered by the sample. In darkfield microscopy, the light scattered by non-target objects should be blocked or minimized to analyze target objects. Light scattering occurs where there is a difference of refractive index between an optical target and its surrounding medium. Reducing refractive index differences by immersing objects in a medium of comparable refractive index can effectively suppress scattering of light by such objects. Transparent polystyrene beads are used as a standard polymeric microspherical substrate for biochemical analysis, e.g., analyte detection assays, and are generally used in an aqueous medium to maintain broad compatibility with biological samples. For imaging, the mismatch in the refractive index between polystyrene and an aqueous medium can lead to light scattering, and it is an object of this disclosure to modulate light scattering by reducing the presence of aqueous films on the surface of polymeric microspheres and improve their response under microscopy.

In some embodiments, a diameter of the microspheres can be about 1 μm to about 100 μm, e.g., about 1 μm to about 100 μm, about 2 μm to about 90 μm, about 3 μm to about 80 μm, about 4 μm to about 70 μm, about 5 μm to about 60 μm, about 6 μm to about 50 μm, about 7 μm to about 40 μm, about 8 μm to about 30 μm, about 9 μm to about 20 μm, or about 10 μm, e.g., about 1 μm, about 2 μm, about 3 μm, about 4 μm, about 5 μm, about 6 μm, about 7 μm, about 8 μm, about 9 μm, about 10 μm, about 11 μm, about 12 μm, about 13 μm, about 14 μm, about 15 μm, about 16 μm, about 17 μm, about 18 μm, about 19 μm, about 20 μm, about 21 μm, about 22 μm, about 23 μm, about 24 μm, about 25 μm, about 26 μm, about 27 μm, about 28 μm, about 29 μm, about 30 μm, about 31 μm, about 32 μm, about 33 μm, about 34 μm, about 35 μm, about 36 μm, about 37 μm, about 38 μm, about 39 μm, about 40 μm, about 41 μm, about 42 μm, about 43 μm, about 44 μm, about 45 μm, about 46 μm, about 47 μm, about 48 μm, about 49 μm, about 50 μm, about 51 μm, about 52 μm, about 53 μm, about 54 μm, about 55 μm, about 56 μm, about 57 μm, about 58 μm, about 59 μm, about 60 μm, about 61 μm, about 62 μm, about 63 μm, about 64 μm, about 65 μm, about 66 μm, about 67 μm, about 68 μm, about 69 μm, about 70 μm, about 71 μm, about 72 μm, about 73 μm, about 74 μm, about 75 μm, about 76 μm, about 77 μm, about 78 μm, about 79 μm, about 80 μm, about 81 μm, about 82 μm, about 83 μm, about 84 μm, about 85 μm, about 86 μm, about 87 μm, about 88 μm, about 89 μm, about 90 μm, about 91 μm, about 92 μm, about 93 μm, about 94 μm, about 95 μm, about 96 μm, about 97 μm, about 98 μm, about 99 μm, or 100 μm in diameter.

Refractive index mismatch can be described by way of a non-limiting example of nanoparticle binding to the surface of polymeric, e.g., polystyrene, microspheres. While polystyrene microspheres are listed as one example of a polymeric microspheres, this is for illustration and any other fluorescent or non-fluorescent polymeric microsphere with suitable biocompatibility can be used. For example, the polymeric microspheres can include fluorochromes, e.g., surface-bound fluorochromes or embedded fluorochromes, e.g., such that the polymeric microspheres are suitable for assays. In some embodiments, biotinylated polystyrene microspheres were reacted with nanoparticles, e.g., streptavidin conjugated gold nanoparticles, in an aqueous medium. In some embodiments, a diameter of the nanoparticles can be about 40 nm to about 150 nm, e.g., about 40 nm to about 60 nm, about 45 nm to about 55 nm, or about 50 nm, about 50 nm to about 100 nm, about 60 nm to about 90 nm, about 70 nm to about 80 nm, about 100 nm to about 130 nm, or about 120 nm to about 150 nm, e.g., about 40 nm, about 41 nm, about 42 nm, about 43 nm, about 44 nm, about 45 nm, about 46 nm, about 47 nm, about 48 nm, about 49 nm, about 50 nm, about 51 nm, about 52 nm, about 53 nm, about 54 nm, about 55 nm, about 56 nm, about 57 nm, about 58 nm, about 59 nm, about 60 nm, about 61 nm, about 62 nm, about 63 nm, about 64 nm, about 65 nm, about 66 nm, about 67 nm, about 68 nm, about 69 nm, about 70 nm, about 71 nm, about 72 nm, about 73 nm, about 74 nm, about 75 nm, about 76 nm, about 77 nm, about 78 nm, about 79 nm, about 80 nm, about 81 nm, about 82 nm, about 83 nm, about 84 nm, about 85 nm, about 86 nm, about 87 nm, about 88 nm, about 89 nm, about 90 nm, about 91 nm, about 92 nm, about 93 nm, about 94 nm, about 95 nm, about 96 nm, about 97 nm, about 98 nm, about 99 nm, about 100 nm in diameter. In some embodiments, the nanoparticles can have a diameter of between 100 nm and 150 nm, e.g., about 101 nm, about 102 nm, about 103 nm, about 104 nm, about 105 nm, about 106 nm, about 107 nm, about 108 nm, about 109 nm, about 110 nm, about 111 nm, about 112 nm, about 113 nm, about 114 nm, about 115 nm, about 116 nm, about 117 nm, about 118 nm, about 119 nm, about 120 nm, about 121 nm, about 122 nm, about 123 nm, about 124 nm, about 125 nm, about 126 nm, about 127 nm, about 128 nm, about 129 nm, about 130 nm, about 131 nm, about 132 nm, about 133 nm, about 134 nm, about 135 nm, about 136 nm, about 137 nm, about 138 nm, about 139 nm, about 140 nm, about 141 nm, about 142 nm, about 143 nm, about 144 nm, about 145 nm, about 146 nm, about 147 nm, about 148 nm, about 149 nm, or about 150 nm. An example of a nanoparticle suitable for methods disclosed herein are gold nanoparticles. While gold nanoparticles are disclosed herein, this disclosure also contemplates other plasmonic metal or metal-like nanoparticles, e.g., silver nanoparticles.

The level of nanoparticle binding to the microsphere surface was assessed by imaging the microspheres under darkfield microscopy and by either visual inspection or computer-aided analysis to determine the number of nanoparticles bound to individual microspheres. Under these conditions, it was known that there was a high level of light scatter from the polystyrene microspheres that completely obscured the light scatter from the surface-bound nanoparticles. Without wishing to be bound by any particular theory, it was believed the root cause for this result was the larger refractive index of polystyrene, e.g., 1.59, compared to the refractive index of water, e.g., 1.33. This refractive index mismatch between polystyrene and water created very high levels of light scatter from the polystyrene microspheres.

A potential solution may be the use of centrifugation to concentrate the polystyrene microspheres and then resuspend them in a fluid that has a refractive index closer to that of polystyrene. Cinnamon bark oil is an example of such a fluid, but there are other biocompatible fluids having a refractive index close to that of polystyrene, including, but not limited to, polyepoxides, oils, e.g., cassia oil (refractive index of 1.59) or anise oil (refractive index of 1.510), esters, e.g., benzyl benzoate (refractive index of 1.568) or ethyl cinnamate (refractive index of 1.559), aromatic liquids, e g, aniline (refractive index of 1.586) or quinoline (refractive index of 1.627), and non-polar solvents, e.g., carbon disulfide (refractive index of 1.63). As disclosed herein, the refractive index of the polystyrene microspheres is 1.59 and the refractive index of cinnamon bark oil is approximately 1.574 to 1.595. Cinnamon bark oil is a close match to the refractive index of polystyrene, but also is a fluid in which biotinylated polystyrene microspheres are miscible. When imaged under cinnamon bark oil, the background light scatter from the polystyrene-aqueous interface was not eliminated and surface bound gold nanoparticles could not be resolved.

Further experimentation showed that a thin film of adherent water persisted on the surfaces of the polystyrene microspheres after centrifugation and before dispersion in the cinnamon bark oil. While the cinnamon oil matched the index of refraction of the polystyrene microspheres, the persistent thin shell of water surrounding each microsphere created a significant index of refraction mismatch, e.g., 1.59 vs 1.33. This refractive index mismatch again created a high level of light scatter that obscured images of polystyrene bead surface bound nanoparticles such as gold nanoparticles.

Additional experimentation showed that removal of the water film and creating closer contact between the polystyrene and the index matching fluid reduced background light scatter, made it possible to clearly image surface bound nanoparticles.

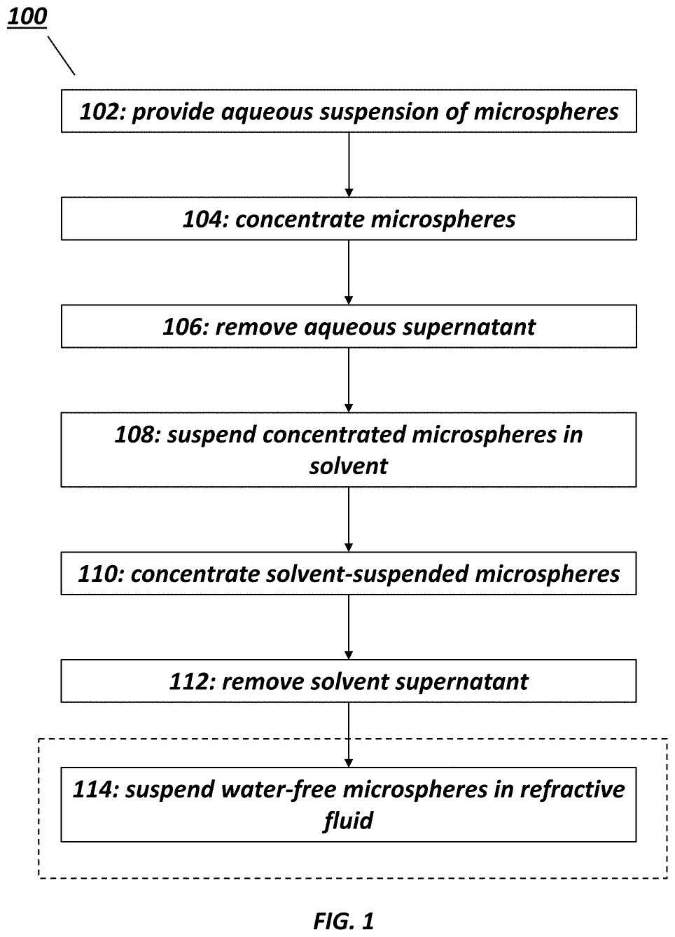

One aspect of this disclosure concerns methods for removal of the water film and creating closer contact between the polystyrene and the index matching fluid. An example method is illustrated in the flow chart of FIG. 1. In the illustrated method 100, the initial aqueous suspension of polystyrene microspheres with surface bound nanoparticles 102 was centrifuged to concentrate the microspheres at step 104 to form a first portion of concentrated microspheres. The aqueous supernatant was removed at step 106 and the microspheres were re-suspended in a solvent, such as a polar solvent, in which water is soluble at step 108.

Solvents suitable for the removal of the residual water film from the microspheres generally should be biocompatible so as to not impact the surface bound species, e.g., antibodies. Suitable solvents include, but are not limited to, alcohols, e.g., ethanol, ketones, e.g., acetone, glycol ethers, e.g., methoxyethanol, alkoxy-substituted hydrocarbons, e.g., dimethoxypropane, and esters, e.g., triethyl phosphate. Other biocompatible solvents suitable for dehydrating the microspheres are further contemplated by this disclosure.

In this example, the polystyrene microsphere—solvent suspension was centrifuged at step 110 to form a second portion of concentrated microspheres and the solvent supernatant removed at step 112. Centrifugation of the polystyrene microsphere—solvent suspension may occur for about 1 second to about 20 minutes, e.g., about 1 second to about 20 seconds, about 5 seconds to about 30 seconds, about 10 seconds to about 40 seconds, about 30 seconds to about 1 minute, about 1 minute to about 20 minutes, about 2 minutes to about 18 minutes, about 3 minutes to about 17 minutes, about 4 minutes to about 16 minutes, about 5 minutes to about 15 minutes, e.g., about 6 minutes to about 14 minutes, about 7 minutes to about 13 minutes, about 8 minutes to about 12 minutes, about 9 minutes to about 11 minutes, or about 10 minutes, e.g., about 1 second, about 2 seconds, about 3 seconds, about 4 seconds, about 5 seconds, about 6 seconds, about 7 seconds, about 8 seconds, about 9 seconds, about 10 seconds, about 11 seconds, about 12 seconds, about 13 seconds, about 14 seconds, about 15 seconds, about 16 seconds, about 17 seconds, about 18 seconds, about 19 seconds, about 20 seconds, about 21 seconds, about 22 seconds, about 23 seconds, about 24 seconds, about 25 seconds, about 26 seconds, about 27 seconds, about 28 seconds, about 29 seconds, about 30 seconds, about 31 seconds, about 32 seconds, about 33 seconds, about 34 seconds, about 35 seconds, about 36 seconds, about 37 seconds, about 38 seconds, about 39 seconds, about 40 seconds, about 41 seconds, about 42 seconds, about 43 seconds, about 44 seconds, about 45 seconds, about 46 seconds, about 47 seconds, about 48 seconds, about 49 seconds, about 50 seconds, about 51 seconds, about 52 seconds, about 53 seconds, about 54 seconds, about 55 seconds, about 56 seconds, about 57 seconds, about 58 seconds, about 59 seconds, about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, or about 20 minutes. With continued reference to FIG. 1, individual concentration steps, e.g., step 104 and 108, can be performed for any suitable length of time disclosed herein and repeated, e.g., 1×, 2×, 3×, or more, to produce the desired water-free microspheres. The now water-free concentrated polystyrene microspheres were re-suspended in an index-matching fluid, including but not limited to, cinnamon bark oil, a polyepoxide, cassia oil, anise oil, aniline, benzyl benzoate, ethyl cinnamate, quinoline, and carbon disulfide at optional step 114. While cinnamon bark oil is listed as one example of a reconstitution fluid, this is for illustration and any other reconstitution fluid that is miscible with the solvent and has suitable biocompatibility and a refractive index can be used.

### EXAMPLES

The function and advantages of these and other embodiments can be better understood from the following examples. These examples are intended to be illustrative in nature and are not considered to be in any way limiting the scope of the invention.

### Example 1

In this example, a comparison of the dark field microscopy images taken of gold nanoparticles attached to the surface of polystyrene microspheres with and without the thin layer of water at the particle surface removed.

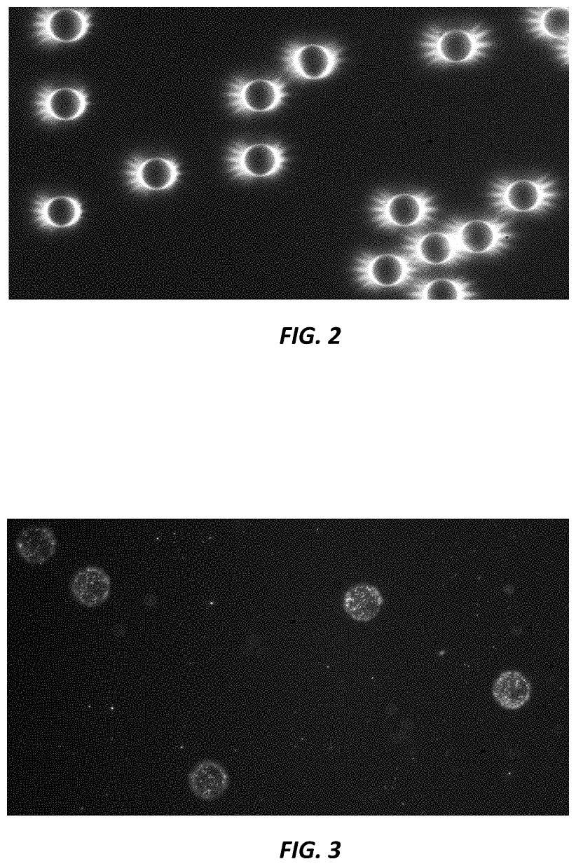

Microspheres with surface bound nanoparticles were produced with the following protocol. Biotin coated polystyrene microspheres suspended in an aqueous medium were purchased from commercial vendors such as Bangs Laboratories, ThermoFisher, and SigmaAldrich. The suspension medium was removed by centrifuging the polystyrene microspheres at 4500×g for 10 minutes, discarding the supernatant and resuspending the spheres in distilled water. Streptavidin-conjugated plasmonic gold nanoparticles were added to the polystyrene microspheres at a ratio of 2000 nanoparticles per polystyrene microsphere. This combination was mixed for five minutes under continuous mixing conditions. The sample was centrifuged at 4500×g for 5 minutes and resuspended in its supernatant. This process was repeated a second time. Following another centrifugation at 4500×g for 5 minutes, the supernatant was removed and the sample reconstituted in the same volume of distilled water. A centrifugation at 4500×g for 10 minutes was performed and the supernatant was removed. The sample was then reconstituted using cinnamon bark oil. For analysis of the polystyrene microspheres without further processing, a microscopy slide was prepared by placing a microscopy spacer on a microscopy slide, adding the recommended volume for the spacer of the sample, and sealing with a cover slip. In FIG. 2, the polystyrene microspheres were suspended in cinnamon bark oil and the residual water film had not been removed. As a consequence, intense background light scatter obscured the surface bound nanoparticles.

To remove residual water from the surface of the polystyrene microspheres prior to reconstitution in cinnamon bark oil, and following another centrifugation at 4500×g for 10 minutes, the supernatant was replaced by the equivalent volume of ethanol (96%), and the sample mixed for 5 minutes under continuous mixing conditions. The sample was then centrifuged at 4500×g for 10 minutes and the supernatant removed. The sample was then reconstituted using cinnamon bark oil. A microscopy slide was prepared by placing a microscopy spacer on a microscopy slide, adding the recommended volume for the spacer of the sample, and sealing with a cover slip.

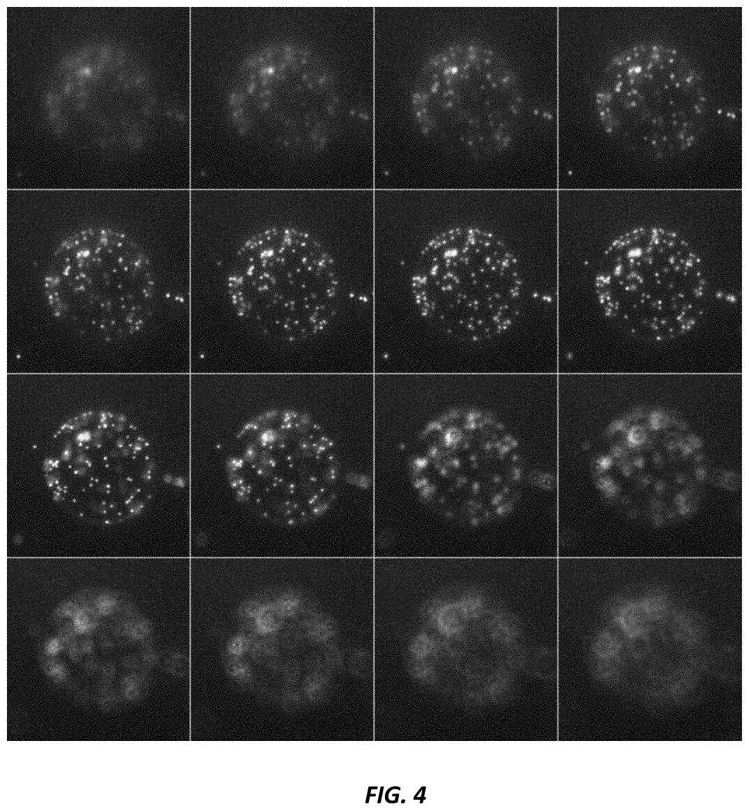

The results of the procedure used in this example are shown as dark field microscope images in FIGS. 3 and 4. In FIG. 3, the water film had been removed, by the steps described above, before the microspheres were suspended in cinnamon bark oil. Background light scatter was substantially reduced or eliminated, and surface bound nanoparticles were clearly resolved as individual points of scattered light randomly distributed over the surface of the polystyrene microspheres.

FIG. 4 shows detailed images of a polystyrene bead immersed in a refractive index-matched medium taken at different Z-planes. The contour of the bead was not visible due to the close match of the surrounding medium's refractive index to that of the polystyrene bead, e.g., approximately 1.59. Surface-bound gold plasmonic nanoparticles were visible from every Z-plane on the entire surface of the polystyrene bead as the bead is transparent and the interior does not scatter light. The location of each surface bound nanoparticle was accurately determined to within 1 micron. This accuracy was limited only by the point spread function of the microscope.

### Example 2

In this example, a control material for plasmonic gold nanoparticle-based optical systems is described.

Gold plasmonic nanoparticles with diameters in the range of 40 nm-100 nm are to be conjugated with monoclonal antibodies that are specific for immune cell surface antigens. When immune cells are reacted with these specific nanoparticles in a biologically compatible aqueous medium, the nanoparticles will bind to the surface of certain immune cells, in proportion to the abundance of the specific surface antigen. These immune cell-bound nanoparticles can be individually resolved under dark field illumination and counted by automated image analysis software. This high-resolution imaging is made possible because there is an index of refraction match between the immune cell and the surrounding aqueous medium, which substantially reduces or eliminates background light scatter caused by refraction and reflections at the cell-water interface. Systems able to analyze cells in the above-mentioned manner require of stable control material to confirm proper functioning.

Since it is difficult and costly to stabilize immune cells, polystyrene microspheres with surface bound gold nanoparticles are perfect candidates as control material for such systems. However, as described in the first example, to be able to see the surface-bound nanoparticles, the microspheres need to be in close contact with the refractive index matched media.

Here, biotin-coated microspheres with surface bound streptavidin coated nanoparticles were prepared according to the procedure of FIG. 1 and used as a control material. The stability of streptavidin coated gold nanoparticles over the period of 52 weeks (i.e., one year) is described.

To produce the biotinylated polystyrene microspheres with surface-bound streptavidin coated gold nanoparticles, biotin coated microspheres were suspended in an aqueous medium had the suspension medium removed by centrifuging the microspheres at 4500×g for 10 minutes, discarding the supernatant and resuspending the spheres in distilled water. Streptavidin-conjugated plasmonic nanoparticles were added to the microspheres at a ratio of 2000 nanoparticles per microsphere. This combination was mixed for five minutes under continuous mixing conditions. The sample was centrifuged at 4500×g for 5 minutes and resuspended in its supernatant. This process was repeated a second time. Another centrifugation at 4500×g for 5 minutes was done, the supernatant removed, and the sample reconstituted in the same volume of distilled water. This process was repeated a second time for a 10 minute centrifugation time. Following another centrifugation at 4500×g for 10 minutes was done, the supernatant was replaced by the equivalent volume of ethanol (96%), and the sample mixed for 5 minutes under continuous mixing conditions. The sample was then centrifuged at 4500×g for 10 minutes and the supernatant removed. The sample was reconstituted using cinnamon bark oil. A microscopy slide was prepared by placing a microscopy spacer on a microscopy slide, adding the recommended volume for the spacer of the sample, and sealing with a cover slip.

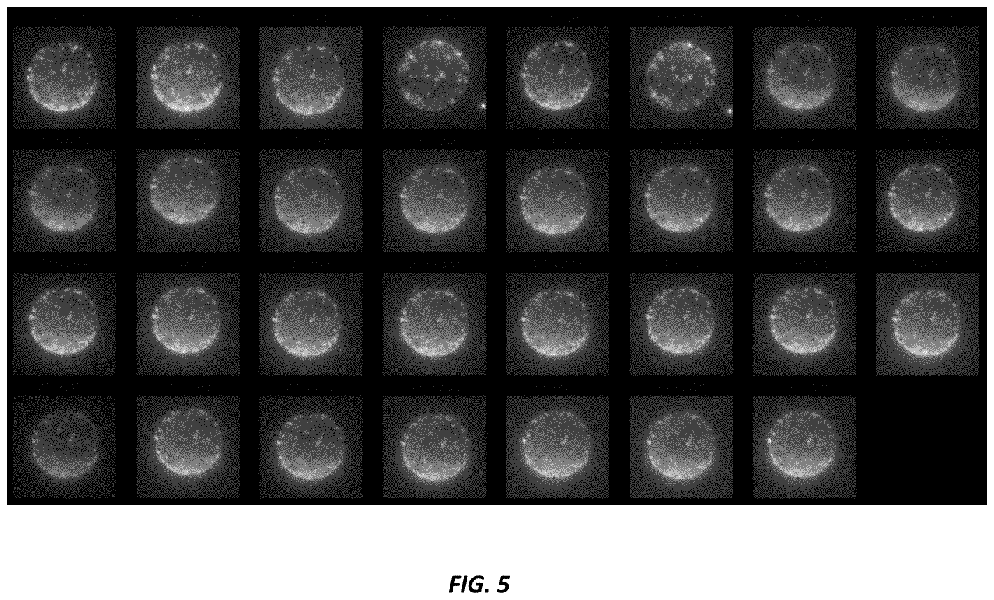

FIG. 5 illustrates maximum intensity projection (MIP) images of a polystyrene microsphere with bound gold nanoparticles immersed in refractive index-matched media imaged between 3 and 52 weeks after production. These results show that the position of the nanoparticles on a given microsphere remained substantially fixed over a period of 52 weeks, demonstrating the production of a stable material suitable for quantitative cell assays.

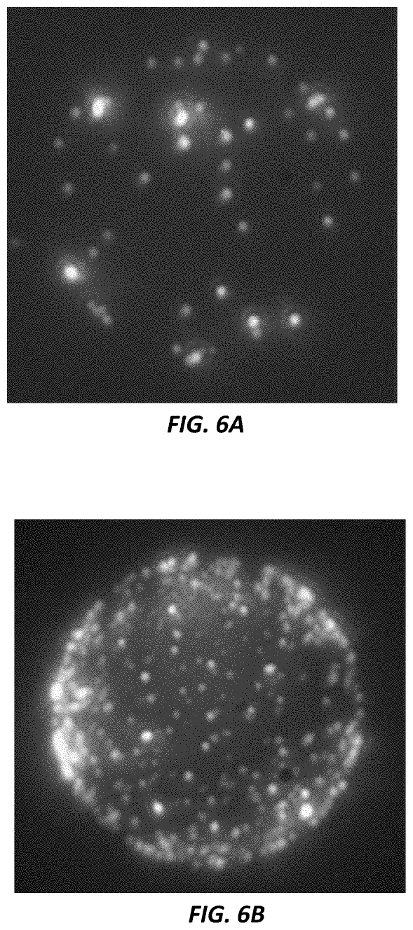

FIGS. 6A-6B illustrate spectroscopic images of a polystyrene microsphere with surface-bound gold nanoparticles immersed in refractive index-matched media at different nanoparticle densities. In FIG. 6A, the ratio of gold nanoparticles to the polystyrene microsphere during the binding reaction was 1000:1 and in FIG. 6B, the ratio of gold nanoparticles to the polystyrene microsphere during the binding reaction was 2000:1.

### Example 3

In this example, multiplexed analysis using fluorescent microspheres, which are readily commercially available in spectrally non-overlapping multiple fluorescent colors, is described. Multiple fluorescent-colored microspheres can also be employed in quality control materials for multiplexed analyses.

Commercially available fluorescent microspheres in a broad range of output colors are to be coated with reactants such as antibodies or nucleic acid strands in an aqueous medium when the intended uses are multiplexed immunoassays, multiplexed DNA or RNA hybridization assays or combinations thereof for the purpose of detecting soluble analytes. After reaction with the desired analytes, the sample is to be incubated with appropriately conjugated gold nanoparticles in an aqueous medium. Next the methods for removal of the water film described herein to create closer contact between the polystyrene microspheres and the index matching fluid are employed as described in detail in Example 1. Following removal of the water film, the sample is to then be resuspended in an index matching fluid and prepared for combined fluorescent and darkfield analysis.

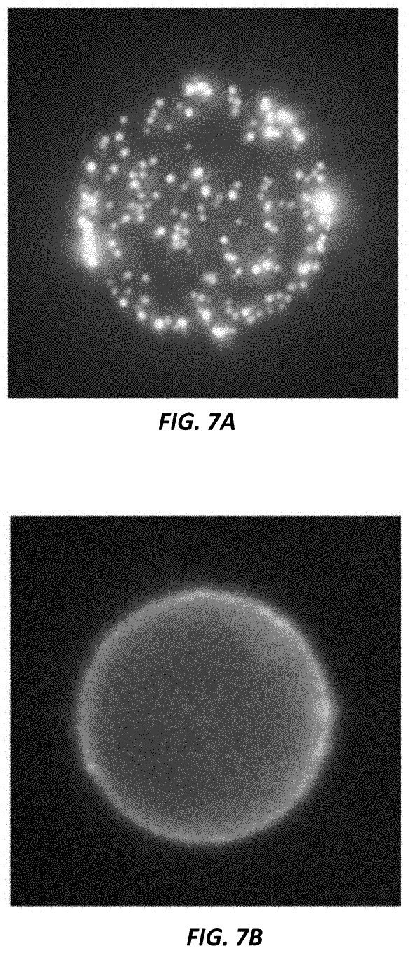

FIGS. 7A-7B illustrates a type of results that may be acquired for fluorescent microspheres. FIGS. 7A-7B illustrate spectroscopic images of a polystyrene microsphere with surface-bound gold nanoparticles immersed in refractive index-matched media. In FIG. 7A, which is a MIP of the polystyrene microsphere with surface-bound gold nanoparticles imaged at different Z-planes under darkfield illumination, the surface-bound gold plasmonic nanoparticles were individually resolved with the location of each surface bound nanoparticle accurately determined within 1 micron. In FIG. 7B, the polystyrene microsphere with surface-bound gold nanoparticles shown in FIG. 7A was imaged using fluorescence microscopy at its equatorial plane.

### Example 4

In this example, the microspheres can also be in other non-spherical shapes, such as micro-rods or similar. If multiplexing with fluorescent microspheres is undesirable multiple shapes of micro particles can also be employed for the purpose of multiplexing as it is described in the third example.

While the present disclosure has explicitly discussed certain particular embodiments and examples of the present disclosure, those skilled in the art will appreciate that the disclosure is not intended to be limited to such embodiments or examples. On the contrary, the present disclosure encompasses various alternatives, modifications, and equivalents of such particular embodiments and/or examples, as will be appreciated by those of skill in the art.

Accordingly, examples, methods and diagrams should not be read as limited to a particular described order or arrangement of steps or elements unless explicitly stated or clearly required from context (e.g., otherwise inoperable). Furthermore, different features of particular elements that may be exemplified in different embodiments may be combined with one another in some embodiments.

The phraseology and terminology used herein is for the purpose of description and should not be regarded as limiting. As used herein, the term “plurality” refers to two or more items or components. The terms “comprising,” “including,” “carrying,” “having,” “containing,” and “involving,” whether in the written description or the claims and the like, are open-ended terms, i.e., to mean “including but not limited to.” Thus, the use of such terms is meant to encompass the items listed thereafter, and equivalents thereof, as well as additional items. Only the transitional phrases “consisting of” and “consisting essentially of,” are closed or semi-closed transitional phrases, respectively, with respect to the claims. Use of ordinal terms such as “first,” “second,” “third,” and the like in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

Having thus described several aspects of at least one embodiment, it is to be appreciated various alterations, modifications, and improvements will readily occur to those skilled in the art. Any feature described in any embodiment may be included in or substituted for any feature of any other embodiment. Such alterations, modifications, and improvements are intended to be part of this disclosure and are intended to be within the scope of the invention. Accordingly, the foregoing description and drawings are by way of example only. Those skilled in the art should appreciate that the parameters and configurations described herein are exemplary and that actual parameters and/or configurations will depend on the specific application in which the disclosed methods and materials are used. Those skilled in the art should also recognize or be able to ascertain, using no more than routine experimentation, equivalents to the specific embodiments disclosed.

What is claimed is:

1. A method of preparing microspheres, comprising:
providing an aqueous suspension of the microspheres;
concentrating the microspheres to form a first portion of concentrated microspheres and an aqueous supernatant;
removing the aqueous supernatant from the first portion of the concentrated microspheres;
suspending the first portion of the concentrated microspheres in a solvent miscible with the aqueous supernatant to form solvent-suspended microspheres;
concentrating the solvent-suspended microspheres to form a second portion of concentrated microspheres and a solvent supernatant; and
removing the solvent supernatant from the second portion of concentrated microspheres, thereby preparing the microspheres.

2. The method of claim 1, further comprising suspending the second portion of concentrated microspheres in a fluid having a refractive index substantially similar to that of a material used to manufacture the microspheres.

3. The method of claim 2, wherein suspending the second portion of concentrated microspheres comprises suspending the second portion of concentrated microspheres in an oil, an ester, an aromatic liquid, a non-polar solvent, or a polyepoxide.

4. The method of claim 1, wherein concentrating the microspheres or the solvent-suspended microspheres occurs under centrifugation.

5. The method of claim 4, wherein concentrating under centrifugation occurs for about 1 second to about 20 minutes.

6. The method of claim 1, wherein the provided microspheres have a diameter from about 1 μm to about 100 μm.

7. The method of claim 6, wherein the provided microspheres comprise surface-bound nanoparticles.

8. The method of claim 6, wherein the provided microspheres comprise surface-bound fluorochromes.

9. The method of claim 6, wherein the provided microspheres comprise embedded fluorochromes.

10. The method of claim 7, wherein the surface-bound nanoparticles of the provided polystyrene microspheres have a diameter from about 40 nm to about 150 nm.

11. The method of claim 1, where providing the aqueous suspension of the microspheres comprises providing an aqueous suspension of polystyrene microspheres.

12. The method of claim 1, wherein suspending the first portion of the concentrated microspheres comprises suspending the first portion of the concentrated microspheres in a solvent selected from alcohols, ketones, glycol ethers, alkoxy-substituted hydrocarbons, and esters.

13. The method of claim 12, wherein the suspending solvent is an alcohol.

14. The method of claim 13, wherein the alcohol is ethanol.

15. A method of preparing microspheres, comprising:
providing an aqueous suspension of the microspheres;
concentrating the microspheres to form a first portion of concentrated microspheres and aqueous supernatant;
removing the aqueous supernatant from the first portion of the concentrated microspheres;
suspending the first portion of the concentrated microspheres in a solvent miscible with the aqueous supernatant to form solvent-suspended microspheres;
concentrating the solvent-suspended microspheres to form a second portion of concentrated microspheres and a solvent supernatant;
removing the solvent supernatant from the second portion of concentrated microspheres, thereby preparing the microspheres; and
suspending the second portion of concentrated microspheres in a fluid having a refractive index substantially similar to that of a material used to manufacture the microspheres.

16. The method of claim 15, wherein suspending the second portion of concentrated microspheres comprises suspending the second portion of concentrated microspheres in in an oil, an ester, an aromatic liquid, a non-polar solvent, or a polyepoxide.

17. The method of claim 15, wherein concentrating the microspheres or the solvent-suspended microspheres occurs under centrifugation.

18. The method of claim 17, wherein concentrating under centrifugation occurs for about 1 second to about 20 minutes.

19. The method of claim 15, wherein the provided microspheres have a diameter from about 1 μm to about 100 μm.

20. The method of claim 15, wherein the provided microspheres comprise surface-bound nanoparticles.

21. The method of claim 20, wherein the surface-bound nanoparticles of the provided polystyrene microspheres have a diameter from about 40 nm to about 150 nm.

22. The method of claim 15, wherein the provided microspheres comprise surface-bound fluorochromes.

23. The method of claim 15, wherein the provided microspheres comprise embedded fluorochromes.

24. The method of claim 15, where providing the aqueous suspension of the microspheres comprises providing an aqueous suspension of polystyrene microspheres.

25. The method of claim 15, wherein suspending the first portion of the concentrated microspheres comprises suspending the first portion of the concentrated microspheres in a solvent selected from alcohols, ketones, glycol ethers, alkoxy-substituted hydrocarbons, and esters.

26. The method of claim 25, wherein the suspending solvent is an alcohol.

27. The method of claim 26, wherein the alcohol is ethanol.
